(19) United States
(12) Patent Application Publication
(10) Pub. No.: US 2017/0296142 A1
(43) Pub. Date: Oct. 19, 2017

(54) ULTRASOUND IMAGING SYSTEM AND METHOD FOR STERILIZING A PROBE

(71) Applicant: General Electric Company, Schenectady, NY (US)

(72) Inventors: Lionel Wodecki, Maisons Alfort (FR); Thomas Andrew Kraus, Waukesha, WI (US)

(21) Appl. No.: 15/131,509

(22) Filed: Apr. 18, 2016

(51) Int. Cl. A61B 8/00; A61L 2/10; A61L 2/28

(52) U.S. Cl. CPC A61B 8/4422; A61L 2/28; A61L 2/10; A61B 8/462; A61B 8/4427; A61B 8/4472; A61B 8/4433; A61B 8/4483

(57) ABSTRACT

An ultrasound imaging system includes a probe adapted to acquire ultrasound data, the probe including a probe head with a plurality of transducer elements, a housing defining a probe storage compartment adapted to receive the probe head, an ultraviolet light source disposed within the housing and configured to irradiate and sterilize the probe head when the probe head is placed in the probe storage compartment, and a screen attached to the housing and configured to display an ultrasound image based on the ultrasound data acquired with the probe. A method of sterilizing a probe includes inserting the probe head into the storage compartment defined by the housing and sterilizing the probe head with the ultraviolet light source.

### FIELD OF THE INVENTION

This disclosure relates generally to an ultrasound imaging system and method for sterilizing a probe. The ultrasound imaging system includes a probe including a probe head including a plurality of transducer elements. The ultrasound imaging system includes a housing defining a probe storage compartment adapted to receive the probe head, and an ultraviolet light source disposed within the housing and configured to irradiate and sterilize the probe head when the probe head is placed in the probe storage compartment. The method for sterilizing the probe includes inserting the probe head into the probe storage compartment and sterilizing the probe head with the ultraviolet light source.

### BACKGROUND OF THE INVENTION

When using an ultrasound imaging system, particularly a system including a wireless probe, it may be difficult to keep the probe associated with the ultrasound imaging system. For example, it is easy for the user to misplace the probe when switching between users, patients, or traveling from one examination site to the next.

Additionally, when acquiring ultrasound images, it is important for patient safety to ensure that the probe is sterilized prior to scanning. Known solutions involve either chemical baths or placing the probe in a dedicated stand-alone ultraviolet (UV) sterilization unit. Neither of these solutions is ideal for an ultrasound imaging system which is often used on more than one patient and in many different locations, some of which might be far from the chemical bath or sterilization unit.

For these and other reasons an improved ultrasound imaging system and method for sterilizing a probe are desired.

### BRIEF DESCRIPTION OF THE INVENTION

The above-mentioned shortcomings, disadvantages and problems are addressed herein which will be understood by reading and understanding the following specification.

In an embodiment, an ultrasound imaging system includes a probe adapted to acquire ultrasound data, the probe including a probe head with a plurality of transducer elements. The ultrasound imaging system includes a housing defining a probe storage compartment adapted to receive the probe head, an ultraviolet light source disposed within the housing and configured to irradiate and sterilize the probe head when the probe head is placed in the probe storage compartment, and a screen attached to the housing. The screen is configured to display an ultrasound image based on the ultrasound data acquired with the probe.

In an embodiment, a method of sterilizing a probe that is a component of an ultrasound imaging system, where the ultrasound imaging system includes a probe including a probe head including a plurality of transducer elements. The ultrasound imaging system includes a housing defining a probe storage compartment adapted to receive the probe head, an ultraviolet light source disposed within the housing and adapted to irradiate the probe head when the probe head is positioned within the probe storage compartment, and a screen attached to the housing. The method includes inserting the probe head into the probe storage compartment and sterilizing the probe head with the ultraviolet light source.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a schematic diagram of an ultrasound imaging system in accordance with an embodiment;

FIG. 3 is a schematic diagram of an ultrasound imaging system in accordance with an embodiment;

FIG. 4 is a schematic diagram of an ultrasound imaging system in accordance with an embodiment;

FIG. 5 is a schematic diagram of an ultrasound imaging system in accordance with an embodiment;

FIG. 6 is a schematic diagram of an ultrasound imaging system in accordance with an embodiment;

FIG. 7 is a schematic diagram of an ultrasound imaging system in accordance with an embodiment; and

FIG. 8 is a schematic diagram of an ultrasound imaging system in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

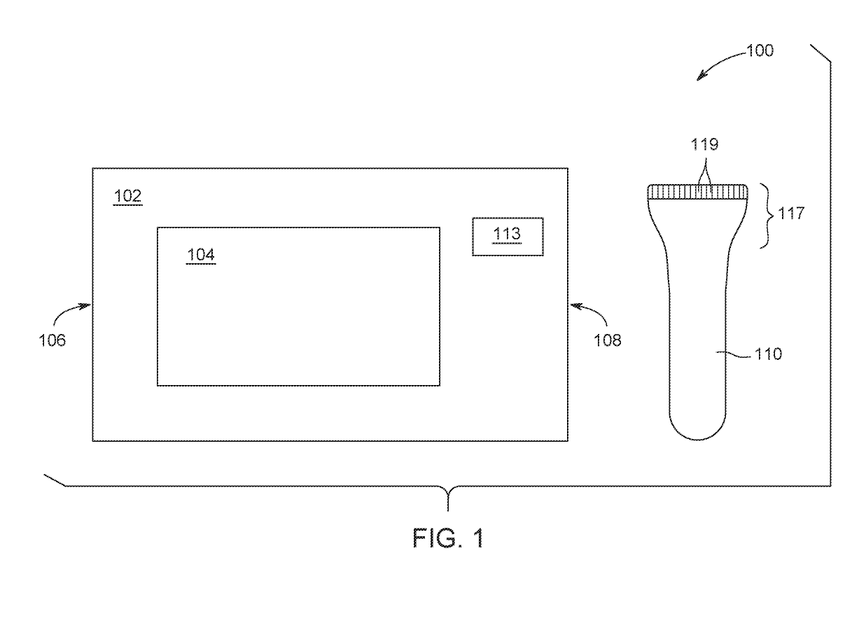

FIG. 1 is a schematic diagram of an ultrasound imaging system 100 in accordance with an embodiment. The ultrasound imaging system 100 includes a housing 102, a screen 104 disposed on an outside surface of the housing 102, and a probe 110. The probe 110 includes a probe head 117 including a plurality of transducer elements 119 arranged in an array. The housing 102 includes a first end 106 and a second end 108 that is opposite of the first end 106. According to an embodiment, the first end 106 may be closed and the second end 108 may be configured to be selectively opened. The housing 102 may be made from any material including: a plastic; a metal, such as aluminum, steel, or an alloy; or a composite material such as fiberglass or carbon fiber. The housing 102 may adapted to be hand-carried, or according to other embodiments, the housing 102 may be part of a laptop or a cart-based ultrasound imaging system.

A processor (not shown in FIG. 1) may also be disposed inside the housing 102. The ultrasound imaging system 100 also includes an ultraviolet light source 113 disposed within the housing 102. The ultraviolet light source 113 is adapted to emit ultraviolet light. The ultraviolet light source 113 will be described in detail hereinafter. The probe 110 may be wired or wireless. The probe 110 is in electronic communication with one or more components, such as the processor inside the housing 102. A plurality of transducer elements 119 are adapted to transmit ultrasonic waves into a subject and receive ultrasound waves from the subject. The probe 110 may be a linear probe, an array probe, a curved array probe, or a 2D array probe. If the probe 110 is a 2D array probe, the probe 110 may be a 1.25D array probe, a 1.5D array probe, a 1.75D array probe, or an E4D probe. According to some embodiments, the probe 110 may contain all of the necessary components to transmit ultrasound waves, receive ultrasound waves, and generate an image based on the received ultrasound waves. For example, the probe 110 may include a separate processor, a transmitter, a transmit beamformer, a receiver, a receive beamformer, and memory or buffer. The probe 110 may acquire ultrasound data and generate ultrasound images, which are then transferred to the housing 102, where the images are displayed on the screen 104. According to other embodiments, some of the components needed to transmit ultrasound waves, receive ultrasound waves, and generate images based on the received ultrasound waves may be disposed in the housing 102. For example, a processor may be disposed in the housing to generate images based on the ultrasound data. According to another embodiment, the transmitter, transmit beamformer, receiver, and receive beamformer may also be disposed in the housing 102. It should be appreciated that the aforementioned elements used to transmit ultrasound waves, receive ultrasound waves, and generate images based on the ultrasound waves may be distributed between the probe 110 and the housing 102 in different manners according to various embodiments.

The transmit beamformer may be a hardware transmit beamformer or transmit beamforming may be performed with software in a processor, a GPU (graphics processing unit), or any other hardware component configured to perform processing operations Likewise, the receive beamformer may be a hardware receive beamformer or the receive beamforming may be performed with software in a processor, a GPU, or any other hardware component configured to perform processing operations. For embodiments where both the transmit beamforming and the receive beamforming are performed in software, a single processor may be used to perform both the transmit beamforming and the receive beamforming, or multiple processors may be used.

The screen 104 is configured for displaying ultrasound images generated based on ultrasound data acquired with the probe 110. According to some embodiments, the screen 104 may be a touch screen or a multi-touch screen. For example, the screen 104 may be configured to receive touch-based inputs and/or gestures from a user to control imaging parameters, to adjust or control display settings, to manipulate acquired images, to control a sterilization process, and the like. While not shown in FIG. 1, other embodiments may include one or more buttons, switches, or other physical controls to control various functions, settings, or parameters on the ultrasound imaging system 100.

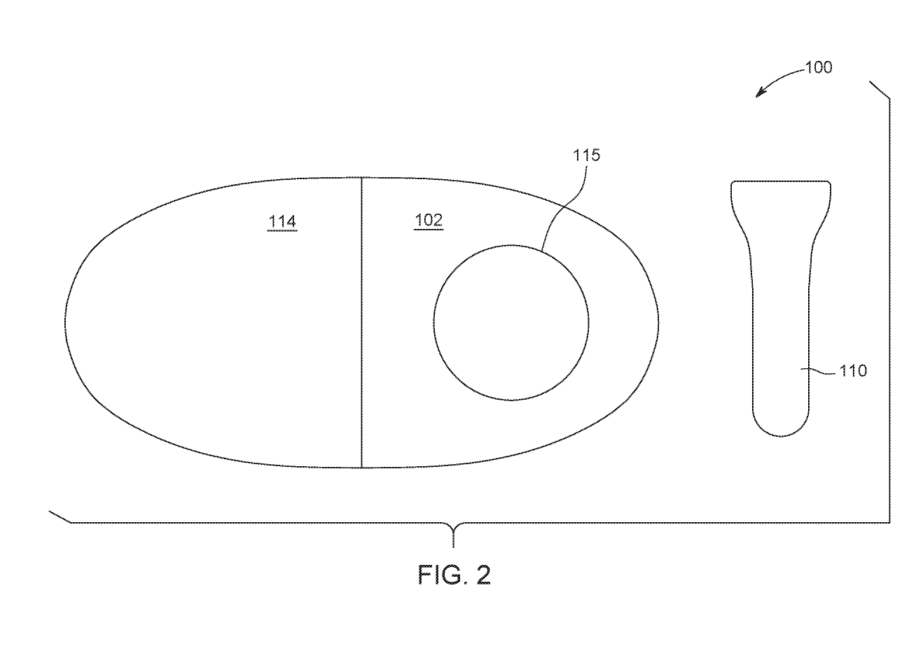

FIG. 2 is a schematic representation of the ultrasound imaging system 100 in accordance with an embodiment. Common reference numbers are used to identify previously described elements. FIG. 2 is an elevational view of the housing 102. The view direction in FIG. 2 is towards the second end 108. As mentioned previously, the second end 108 is configured to be selectively opened. The second end 108 in FIG. 2 is shown in an open position. The housing 102 defines a probe storage compartment 115. According to an embodiment, the second end 108 may have an access panel 114 connected to the housing 102 with a hinge or pivot that allows the access panel 114 to be positioned in either a closed position or an open position. The access panel 114 is shown in the open position in FIG. 2. The second end 108 may also be selectively opened with other mechanisms according to other embodiments. For example, the probe access panel 114 may be positioned in a track within the housing 102, allowing the probe access panel 114 to slide between an open position and a closed position. In some embodiments, the probe access panel 114 may be detachable from the housing 102.

The probe storage compartment 115 may be sized to completely enclose the probe 110 when the probe 110 is inserted in the probe storage compartment and the probe access panel 114 is closed. The probe storage compartment 115 has a circular cross section according to the embodiment shown in FIG. 2, but it should be appreciated that the housing 102 may be shaped differently according to other embodiments. For example, the probe storage compartment 115 may be a female shape that complements the shape of the probe 110 in order to hold the probe 110 securely in the probe storage compartment 115.

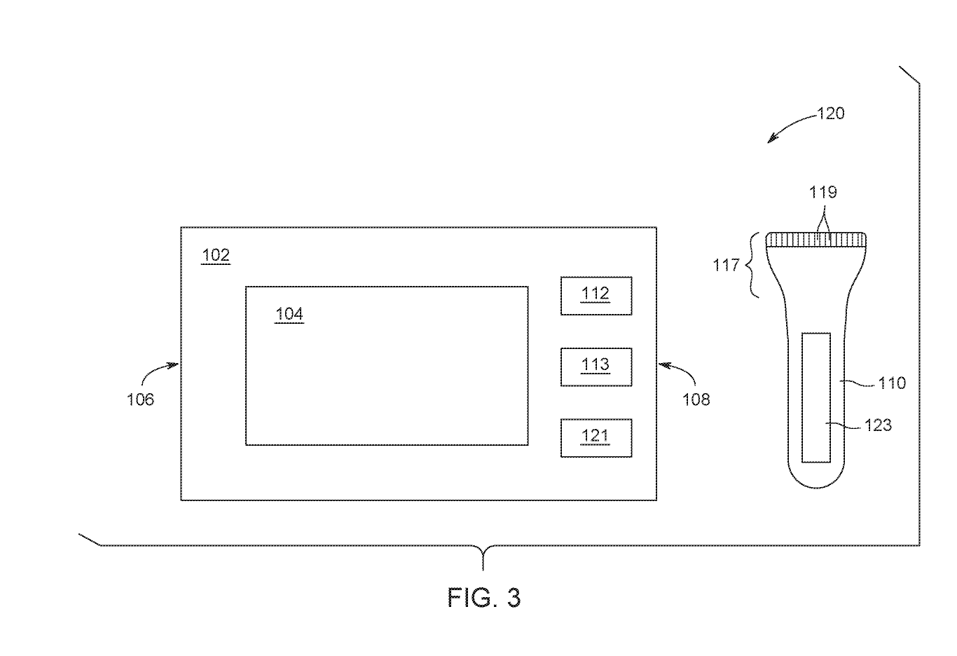

FIG. 3 is a schematic representation of an ultrasound imaging system 120 in accordance with an embodiment. Common reference numbers are used to identify elements that were previously described with respect to FIGS. 1 and 2. The ultrasound imaging system 120 includes the housing 102, the screen 104 disposed on the housing, and the probe 110. As with the embodiment described with respect to FIGS. 1 and 2, the housing 102 has a first end 106 that is closed and a second end 108 that is configured to be selectively opened. The ultrasound imaging system 120 includes the probe 110. The ultrasound imaging system 120 also includes a sterilization status indicator 112 and the ultraviolet light source 113. The ultrasound imaging system 120 includes a charging module 121 that is adapted to recharge the probe 110. It should be apparent to those skilled in the art that the charging module 121 is the most useful for embodiments where the probe 110 is a wireless probe. As described previously, the housing 102 defines a probe storage compartment (not shown in FIG. 3). The ultraviolet light source 113 is disposed within the housing 102 and adapted to irradiate the probe storage compartment 115 with ultraviolet light. When the probe 110 is positioned in the probe storage compartment 115, the ultraviolet light source 113 may irradiate the probe 110 with ultraviolet light. The ultraviolet light therefore sterilizes the probe 110 while the probe 110 is placed in the probe storage compartment 115.

The ultraviolet light source 113 may be any device configured to emit ultraviolet light, such as an ultraviolet LED (light emitting diode), a gas-discharge lamp, a short-wave ultraviolet lamp, an ultraviolet laser, or any other ultraviolet emitter. The ultraviolet light source 113 may include a plurality of discrete ultraviolet emitters. For example, an embodiment may include multiple ultraviolet LEDs. Embodiments may use a plurality of discrete ultraviolet emitters to ensure that all surfaces of the probe 110 are exposed to ultraviolet light and thus sterilized.

The charging module 121 is disposed within the housing 102 and adapted to recharge the probe 110 when the probe 110 is positioned in the probe storage compartment 115. According to an embodiment, the charging module 121 may include an inductive charger. The inductive charger is capable of recharging a battery 123 within the probe 110 without making physical contact with the probe 110. This advantageously provides the ability to recharge the probe 110 without first securing a physical connection to the charging module 121, which may make it easier for a user to quickly insert the probe 110 into the probe storage compartment 115 or remove the probe 110 from the probe storage compartment 115. According to another embodiment, the charging module 121 may include a magnetic connector that is configured to connect to either a magnetic connector of opposite polarity on the probe 110 or a metal connection surface on the probe 110. The magnetic connector provides an easy way for a user to quickly and easily insert and/or remove the probe 110 from the probe storage compartment 115. According to another embodiment, the charging module 121 may include a plug that is adapted to interface with a compatible connector on the probe 110. The plug of the charging module 121 may be of male or female design. The charging module 121 is connected to a battery or other power supply located within the housing 102 in order to provide the power to recharge the probe battery 123 when the probe 110 is positioned in the probe storage compartment 115.

The sterilization status indicator 112 provides an indication of the sterilization status of the probe 110 when the probe 110 is in the probe storage compartment 115. In some embodiments, the sterilization status indicator 112 may also provide an indication of how close the sterilization process is to being completed. The sterilization status indicator 112 may, for instance include one or more LEDs that indicate the sterilization status. The LEDs, or any other light source, may, for instance, use their activation status (i.e., whether the light is on or off), color, or a combination of activation status and color to indicate the probe sterilization status. In some embodiments, the sterilization status indicator 112 may be displayed on the screen 104. For example, a portion of the screen 104 may be dedicated to displaying the sterilization status when the probe 110 is placed in the probe storage compartment 115. In other embodiments, the sterilization status may only be displayed on the screen 104 in response to user request to see the status. The sterilization status indicator displayed on the screen 102 may be configured to display different colors, icons, text strings, or a combination of colors, icons, and text strings in order to indicate the various sterilization statuses. The sterilization status indicator 112 will be described in additional detail hereinafter.

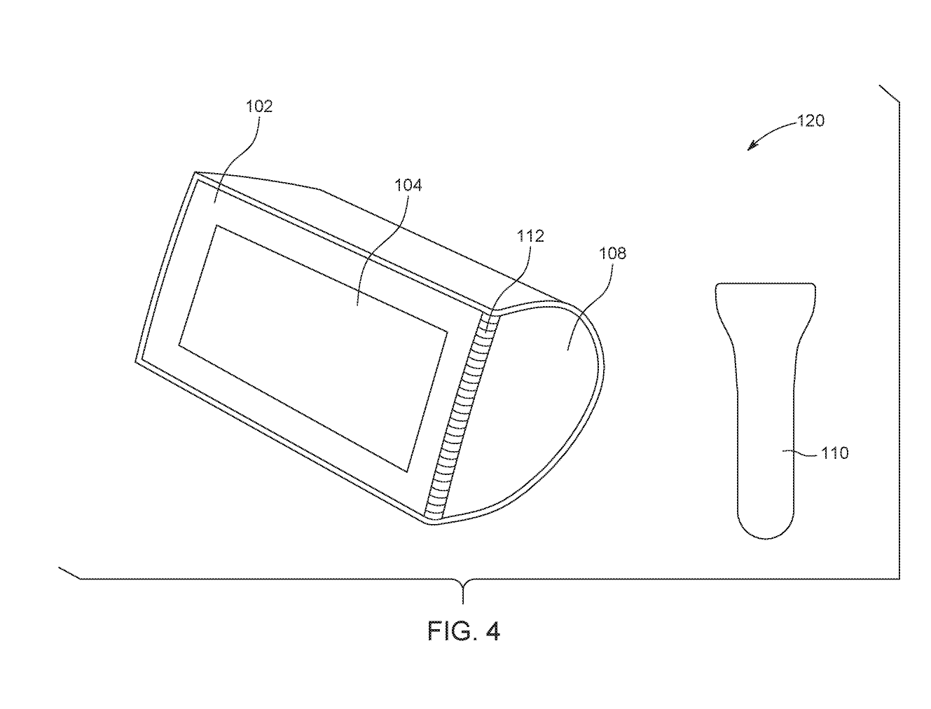

FIG. 4 is a schematic representation of a perspective view of the ultrasound imaging system 120 in accordance with an exemplary embodiment. The second end 108 and the screen 104 are both visible in the perspective view. The sterilization status indicator 112 may comprise a plurality of LEDs arranged in a row according to an embodiment. The sterilization status indicator 112 may be used to show one or more of the following statuses: that the probe 110 is in the probe storage compartment 115 (not shown in FIG. 4), that sterilization of the probe 110 is in-process, and that sterilization of the probe 110 has been completed. The following examples will be described with respect to an exemplary embodiment where the sterilization status indicator 112 includes a plurality of LEDs arranged in a row. According to an exemplary embodiment, the plurality of LEDs may emit a first pattern or color to indicate that the probe 110 is in the probe storage compartment 115. For example, some or all of the LEDs may emit a first color, such as red. The plurality of LEDs may emit a second pattern or color to indicate that sterilization is in-process. According to an embodiment, the LEDs may provide a pulsing or strobing effect to indicate that sterilization is in-process. One example of a pulsing effect is to sequentially illuminate the plurality of LEDs arranged in the row and then sequentially turn off the LEDs in the row. The LEDs may be iteratively illuminated sequentially and then turned off sequentially a number of times in order to provide a pulsing or strobing effect. According to another embodiment, the LEDs arranged in the row may be used to show how close the sterilization process is to being completed. For example, only one LED or a few LEDs may illuminate at the start of the sterilization process and additional LEDs may be illuminated in order to indicate that the sterilization process is closer to being completed. The user would thus be able to tell the status of the sterilization process based on the number of LEDs illuminated in the sterilization status indicator 112 Likewise, if only a few of the LEDs were illuminated, the user would be able to quickly determine that the sterilization process still has a significant amount of time remaining before completion.

The plurality of LEDs may emit a third pattern or color to indicate that the sterilization process has been completed. For example, according to an embodiment, the plurality of LEDs may be illuminated in a single color such as green, for example, to indicate that sterilization of the probe 110 has been completed. It should be appreciated that the colors and patterns associated with indicating that the probe 110 is in the probe storage compartment 115, indicating that sterilization is in-process, and indicating that sterilization has been completed are just exemplary embodiments. Other embodiments using a plurality of LEDs as the sterilization status indicator 112 may use a different combination of colors and/or patterns to indicate various sterilization statuses.

According to an embodiment, the sterilization status indicator 112 may also be used to show a probe charge status. For example, according to an embodiment where the sterilization status indicator 112 comprises a plurality of LEDs, the plurality of LEDs may display the charge status of the probe 110 through the use of additional patterns or colors. According to an embodiment, the sterilization status indicator 112 may display information indicating the capacity of the battery 123 in real-time when the probe 110 is positioned in the probe storage compartment 115.

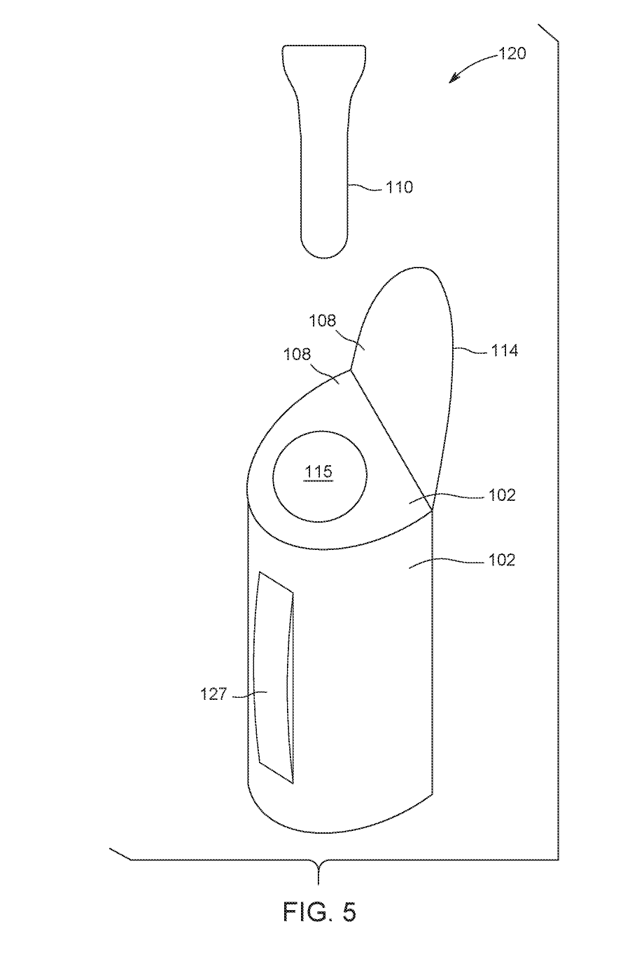

FIG. 5 is a schematic representation of a perspective view the ultrasound imaging system 120 in accordance with an embodiment. The second end 108 is visible in FIG. 5. The probe access panel 114 is shown in an open position, exposing the probe storage compartment 115 defined by the housing 102. The probe 110 may be inserted into the probe storage compartment 115 and then probe access panel 114 may be closed, thus securing the probe 110 in the housing 102 for storage and transport. A strap 127 is visible in the perspective view shown in FIG. 5. The strap 127 may be configured to be worn around a user's hand. The strap 127 may allow for the user to securely hold and carry the housing 102 of the ultrasound imaging system 120. All of the other elements shown in FIG. 5 have been described previously, and will therefore not be described in additional detail.

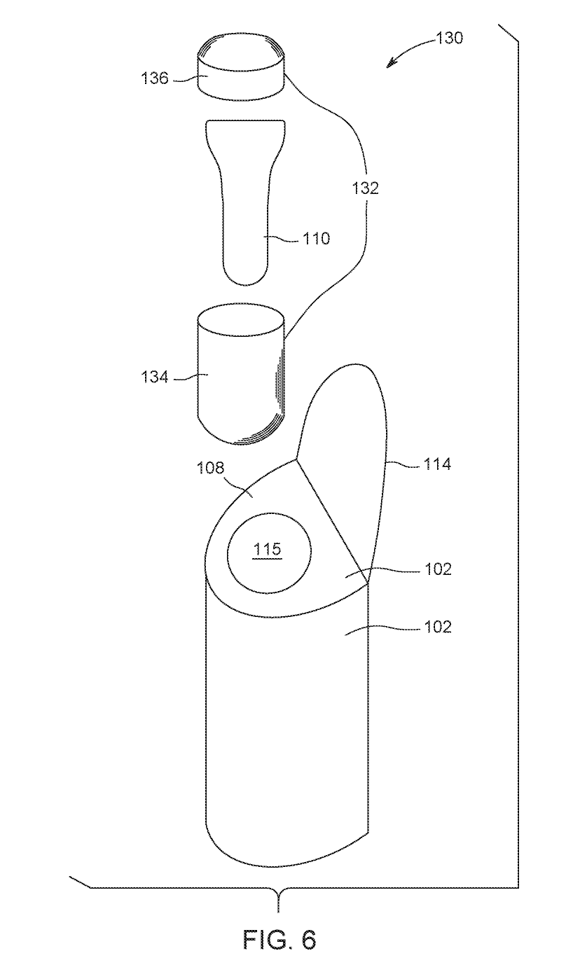

FIG. 6 is a schematic representation of a perspective view of an ultrasound imaging system 130 according to an exemplary embodiment. Many of the elements shown in FIG. 6 are identical to elements that were previously described with respect to earlier figures. Common reference numbers are used to identify elements that are substantially identical to previously described elements. In addition to the elements that were previously described, the ultrasound imaging system 130 includes a removable capsule 132. The removable capsule 132 is adapted to enclose the probe 110. According to an embodiment, the removable capsule 132 includes a top portion 136 and a bottom portion 134. According to the embodiment shown in FIG. 6, the probe storage compartment 115 is adapted to receive the removable capsule 132, and in turn, the probe 110 when the probe 110 is placed in the removable capsule 132.

The removable capsule 132 may be made of any material that is either transparent or translucent with respect to ultraviolet light. For example, the removable capsule 132 may be made from a plastic or from non-polarized glass. It may be beneficial for the removable capsule 132 to be made from a plastic rather than glass for improved impact resistance.

The top portion 136 may be configured to connect or mate with the lower portion 134. Any type of connection mechanism may be used including a threaded connector, a snap-fit connector, latches, etc. The inside of the removable capsule 132 may be shaped or contoured to securely hold the probe 110 in a fixed position. This may advantageously protect the probe 110 while transporting the ultrasound imaging system 130. Additionally, the removable capsule 132 provides the user with an easy way to keep the system clean. If the probe 110 has any dirt or other contaminates on it, the removable capsule 132 is easy to remove and clean, and it keeps dirt and contamination away from the system. Any dirt or contaminates that were on the probe 110 would be captured within the capsule 132, instead of becoming lodged within the probe storage compartment 115. Since the removable capsule 132 is easily removed, the user can simply remove and easily clean the removable capsule 132. Otherwise, it could be very difficult to clean the inside surface of the probe storage compartment 115.

The probe storage compartment 115 provides the user with an extremely convenient way to store, carry, and sterilize the probe 110. This is particularly useful when the probe 110 is a wireless probe configured to wirelessly transmit ultrasound data. Since the ultrasound imaging system 130 is a portable system that is adapted to be hand-carried, it is likely that users will carry the ultrasound imaging system to multiple different locations to examine various patients. As such, the probe storage compartment 115 provides the user with a location to safely and securely store the probe 110 during transportation and/or storage of the ultrasound imaging system.

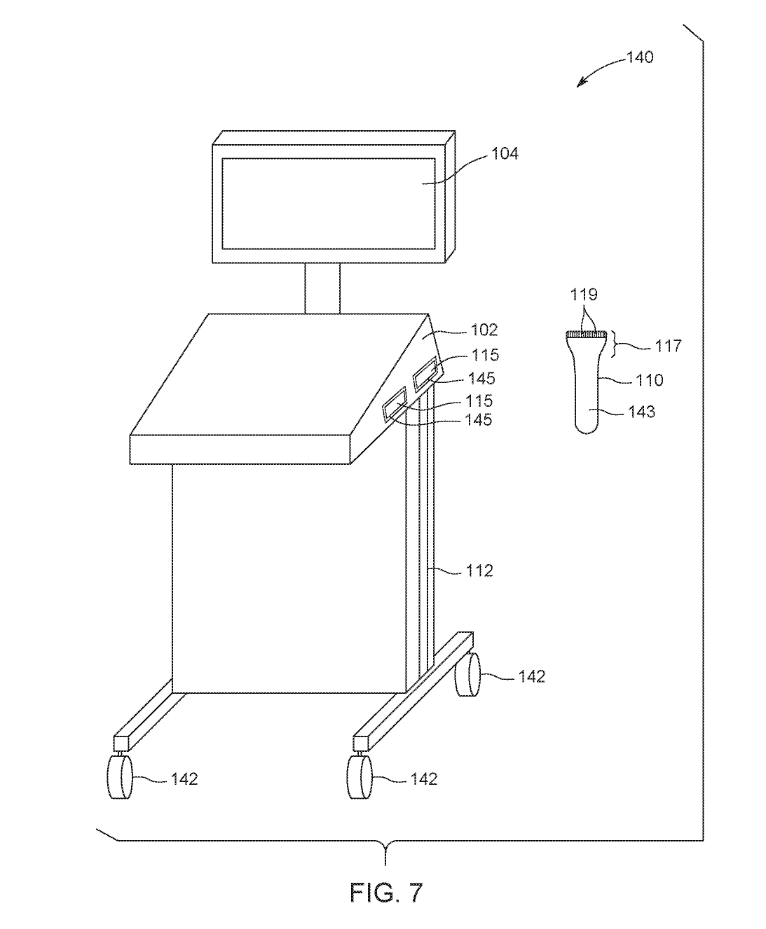

FIG. 7 is a schematic representation of an ultrasound imaging system 140 in accordance with an embodiment. FIG. 7 is a perspective view of the ultrasound imaging system 140. Common reference numbers are used to identify previously described components.

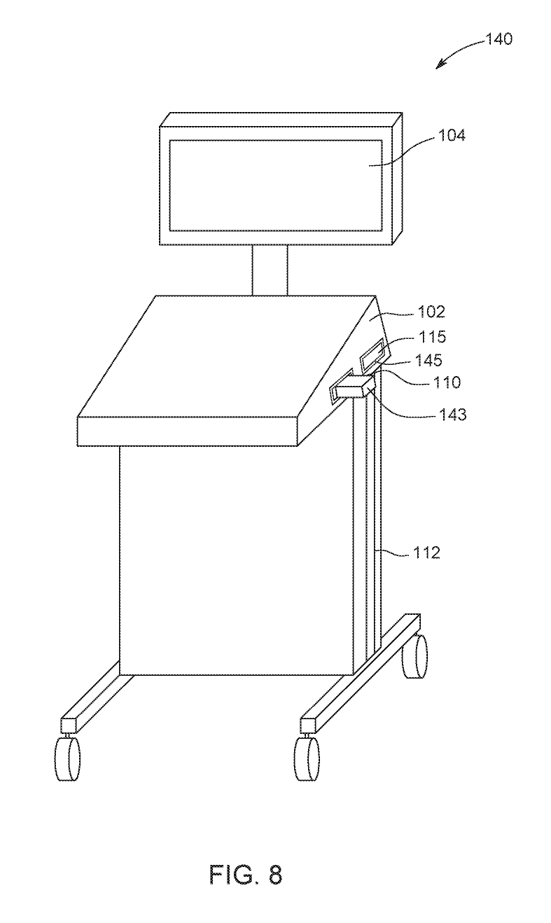

The ultrasound imaging system 140 is a cart-based system and includes the housing 102, the screen 104, and the probe 110. The screen 104 is attached to the housing 102. The housing 102 is attached to wheels 142 to make transporting the ultrasound imaging system 140 easier. The housing 102 defines a plurality of probe storage compartments 115 according to an embodiment. The probe storage compartments 115 may each be sized to fit the entire probe 110, or each probe storage compartment 115 may be configured to receive the probe head 117. There is an ultraviolet light source disposed within the housing 102 and configured to illuminate the probe head 117 when the probe 110 is positioned in the respective probe storage compartment 115. FIG. 8 is a schematic representation of the ultrasound imaging system 140 shown in FIG. 7. The probe 110 is shown positioned in the probe storage compartment 115. As seen in FIG. 8, the probe head 117 is positioned inside the probe storage compartment 115, but a probe handle 143 protrudes from the housing 102. According to other embodiments, the probe 110 may be completely enclosed by the housing 102 when the probe 115 is positioned in the probe storage compartment 115. Additionally, embodiments may include a probe access panel enabling the user to completely enclose the probe 110 in the probe storage compartment 115.

The ultrasound imaging system 140 may include a removable capsule similar to that which was described with respect to FIGS. 5 and 6. The removable capsule may be adapted to receive the probe 110 and fit into the probe storage compartment 115 defined by the housing 102. Or, according to the embodiment shown in FIGS. 7 and 8, the ultrasound imaging system 140 may include a removable sleeve 145. The removable sleeve 145 is made from a material that is either translucent or transparent to ultraviolet light and the removable sleeve 145 is adapted to be easily removed from the probe storage compartment 115 defined by the housing 102. The removable sleeve 145 is configured to secure the probe head 117 within the probe storage compartment 115. The user may remove the removable sleeve 145 for easy cleaning of dirt or other contaminants from the probe 110. While the ultrasound imaging system 140 includes two probe storage compartments 115, it should be appreciated that other embodiments may have only one probe storage compartment 115 or more than two probe storage compartments 115.

The ultrasound imaging system 140 also includes the sterilization status indicator 112. The sterilization status indicator 112 may be a light that illuminates to indicate how close to completion the sterilization process is. The user may, for instance, determine the sterilization status based on how much of the sterilization status indicator 112 is illuminated. However other embodiments may use different types of sterilization status indicators, including any of the variations discussed previously.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An ultrasound imaging system comprising:
a probe adapted to acquire ultrasound data, the probe comprising a probe head with a plurality of transducer elements;
a housing defining a probe storage compartment adapted to receive the probe head;
an ultraviolet light source disposed within the housing and configured to irradiate and sterilize the probe head when the probe head is placed in the probe storage compartment; and
a screen attached to the housing, the screen configured to display an ultrasound image based on the ultrasound data acquired with the probe.

2. The ultrasound imaging system of claim 1, wherein the probe comprises a wireless ultrasound probe.

3. The ultrasound imaging system of claim 2, further comprising a removable capsule adapted to be received within the probe storage compartment, wherein the removable capsule is adapted to enclose the probe.

4. The ultrasound imaging system of claim 3, where the removable capsule is either translucent or transparent with respect to ultraviolet light.

5. The ultrasound imaging system of claim 4, wherein the removable capsule comprises a top portion and a bottom portion and wherein the top portion is adapted to be connected to the bottom portion.

6. The ultrasound imaging system of claim 2, further comprising a sterilization status indicator.

7. The ultrasound imaging system of claim 6, wherein the sterilization status indicator is displayed on the screen.

8. The ultrasound imaging system of claim 6, wherein the sterilization status indicator comprises a plurality of LEDs attached to the housing.

9. The ultrasound imaging system of claim 8, wherein the plurality of LEDs are illuminated in a first pattern to indicate that sterilization is in-process and a second pattern to indicate that sterilization has been completed.

10. The ultrasound imaging system of claim 1, wherein the ultrasound imaging system comprises a hand-carried ultrasound system and the display is disposed on the housing.

11. The ultrasound imaging system of claim 1, wherein the probe storage compartment defined by the housing comprises a first end that is closed and a second end, opposite of the first end, which is configured to be selectively opened, and wherein the probe storage compartment is configured to completely enclose the probe when the second end is closed.

12. The ultrasound imaging system of claim 2, further comprising a charging module adapted to recharge the probe while the probe is positioned in the probe storage compartment.

13. The ultrasound imaging system of claim 12, wherein the charging module comprises an inductive charger.

14. The ultrasound imaging system of claim 12, wherein the charging module comprises a magnetic connector configured to magnetically attach to the probe when the probe is positioned in the probe storage compartment.

15. The ultrasound imaging system of claim 12, wherein the charging module comprises a plug adapted to interface with the probe when the probe is positioned in the probe storage compartment.

16. The ultrasound imaging system of claim 12, further comprising a sterilization status indicator configured to display both a sterilization status and a probe charge status.

17. The ultrasound imaging system of claim 16, wherein the sterilization status indicator comprises a plurality of LEDs.

18. A method of sterilizing a probe that is a component of an ultrasound imaging system, the ultrasound imaging system comprising:
a probe comprising a probe head including a plurality of transducer elements;
a housing defining a probe storage compartment adapted to receive the probe head;
a ultraviolet light source disposed within the housing and adapted to irradiate the probe head when the probe head is positioned within the probe storage compartment; and
a screen attached to the housing;
where the method comprises:
inserting the probe head into the probe storage compartment defined by the housing; and
sterilizing the probe head with the ultraviolet light source.

19. The method of claim 18, further comprising placing the probe into a removable capsule before inserting the probe into the storage compartment, and wherein inserting the probe into the storage compartment comprises inserting both the removable capsule and the probe into the storage compartment.

20. The method of claim 18, further comprising recharging the probe with a charging module while the probe is inserted into the probe storage compartment.
